(19) United States
(12) Patent Application Publication
(10) Pub. No.: US 2024/0181218 A1
(43) Pub. Date: Jun. 6, 2024

(54) Catheter Retention Device

(71) Applicant: Emory University, Atlanta, GA (US)

(72) Inventors: Tyler Joseph Kristoff, Atlanta, GA (US); Vivian Truong, Atlanta, GA (US); Caitlyn Beals, Atlanta, GA (US); Brenae Nelson, Atlanta, GA (US); Anh Thi Ngoc Phan, Atlanta, GA (US); Amenda Mama, Atlanta, GA (US)

(21) Appl. No.: 18/527,046

(22) Filed: Dec. 1, 2023

Related U.S. Application Data
(60) Provisional application No. 63/430,082, filed on Dec. 5, 2022.

(51) Int. Cl. A61M 25/02

(52) U.S. Cl. CPC A61M 25/02; A61M 2025/0266

(57) ABSTRACT

The devices can temporarily secure a catheter to the patient's abdomen thereby minimizing potential complications associated with the procedure. The device may include an expandable support frame. The frame may include a plurality of members. The plurality of members may include a base member, an upper member and a plurality of middle members disposed between the base member and the upper member. The frame may be configured to linearly extend to an expanded state from a collapsed state with respect to the base member so that the plurality of middle members and the upper member are disposed above the base member when in the expanded state. The device may also include a retention member disposed on the upper member. The retention member may be configured to securely hold and surround a medical article. The device may further include an anchor member disposed on the base member.

### CROSS-REFERENCE TO RELATED APPLICATIONS

This application claims the benefit of U.S. Provisional Application No. 63/430,082 filed Dec. 5, 2022. The entirety of this application is hereby incorporated by reference for all purposes.

### BACKGROUND

Numerous conditions, such as liver cirrhosis, can require removal of fluid from the abdominal cavity for analysis, for relief of a condition, such as pressure-driven abdominal discomfort and respiratory distress, among others.

To remove the fluid, for example, by performing a paracentesis, a clinician introduces a catheter into the abdominal cavity and draws fluid. The procedure generally requires the clinician to stabilize the catheter throughout the procedure to prevent dislodgement, taking time and attention away from other matters taking place during the drainage. When a catheter is unintentionally dislodged from the abdomen, it may result in the need for reinsertion of a new catheter which can increase patient discomfort, risk of infection, waste, and cost, as well as further perforation of tissue.

### SUMMARY

Thus, there is a need for a catheter retention device that can retain a catheter during a procedure, such as paracentesis, to prevent dislodgement, thereby avoiding the need for a reinsertion procedure.

Techniques disclosed herein relate generally to devices that can temporarily secure a catheter to the patient's abdomen thereby minimizing potential complications associated with the procedure. The devices can be attached after insertion of the catheter and adjusted to various abdominal tissue morphologies. The devices can allow for a clinician to fixate the catheter at constant depth and insertion angle without the need to hold the catheter in place, ensuring hands-free retention.

The disclosed embodiments may include a catheter retention device. In some embodiments, a device may include an expandable support frame. The frame may include a plurality of members. The plurality of members may include a base member, an upper member and a plurality of middle members disposed between the base member and the upper member. The frame may be configured to linearly extend to an expanded state from a collapsed state with respect to the base member so that the plurality of middle members and the upper member are disposed above the base member when in the expanded state. The device may also include a retention member disposed on the upper member. The retention member may be configured to securely hold and surround a medical article. The device may further include an anchor member disposed on the base member. The anchor member may include an adhesive layer disposed on a bottom face of the anchor member.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, the emphasis being placed upon illustrating the principles of the disclosure.

FIG. 1 shows an example of a catheter retention device in an expanded state according to embodiments.

FIG. 2 shows a partial, enlarged view of the retention member shown in FIG. 1.

FIG. 3 shows the expandable support frame shown in FIG. 1 in a collapsed state.

FIG. 4 shows another example of the catheter retention device in an expanded state according to embodiments.

FIGS. 5-8 shows an illustrative example of a procedure using the catheter retention device according to embodiments. FIG. 5 illustrates an example of a paracentesis catheter after insertion and positioning. FIG. 6 illustrates an example of the catheter retention device locked onto the catheter in a collapsed state. FIG. 7 illustrates an example of the catheter retention device locked onto the catheter in an expanded state. FIG. 8 illustrates an example of the expanded catheter retention device locked onto the catheter affixed to an abdomen of a patient.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

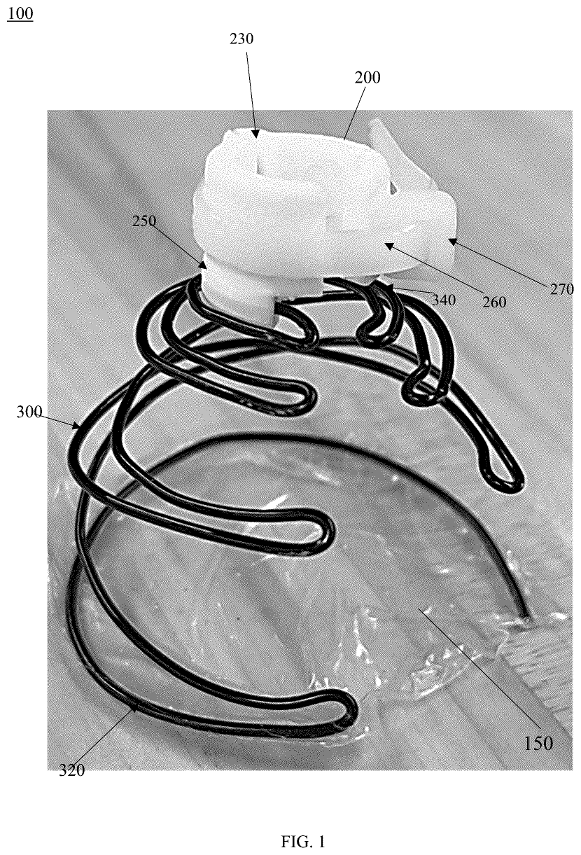

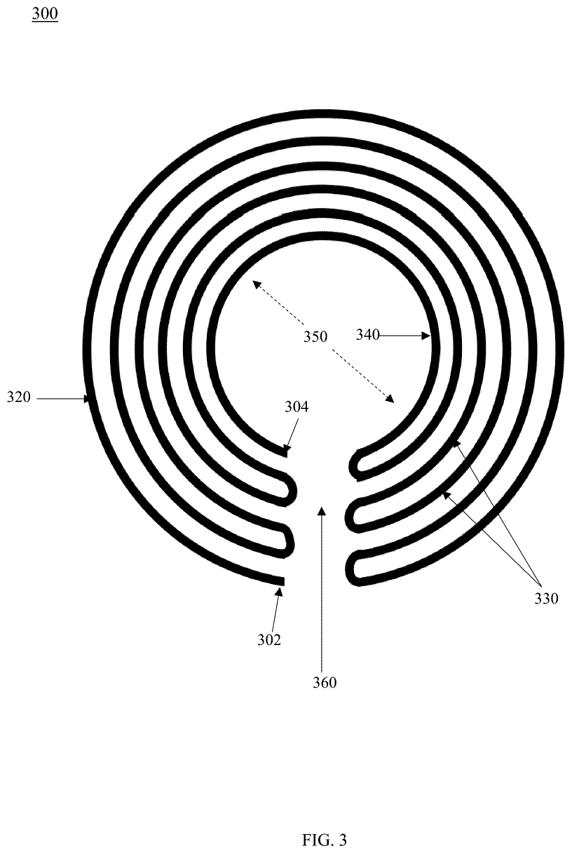

FIGS. 1-4 show examples of a catheter retention device configured to securely hold a medical article, such as a catheter, for a procedure according to embodiments. FIGS. 1-3 show an example of a catheter retention device 100 according to embodiments. As shown in FIG. 1, the device 100 may include an anchor member 150 configured to removably adhere to skin of a patient at an area of insertion of a medical article (e.g., abdomen of the patient); a retention member 200 configured to securely hold and surround a medical article, such as a catheter; and an expandable support frame 300 configured to move from a collapsed, resting, state, for example, as shown in FIG. 3, to an expanded state, for example, as shown in FIG. 1.

In some examples, the anchor member 150 may include a laminate structure with one or more layers. In some examples, the anchor member 150 may include at least an upper layer and a lower layer. In some examples, the upper layer may be a plastic layer, a paper layer, a foam layer (e.g., closed-cell polyethylene foam), among others, or any combination thereof. In some examples, the lower layer may include an adhesive. The lower layer may constitute a lower surface of the anchor member 150 and may include a bottom face configured to contact skin of a patient. In some examples, the lower layer may include a medical-grade adhesive on at least its bottom face and can be either diaphoretic or nondiaphoretic, depending upon the particular application. In some examples, the bottom face may be covered with a backing material. In some examples, the anchor member 150 may be made of one or more materials including but not limited to Tegaderm™.

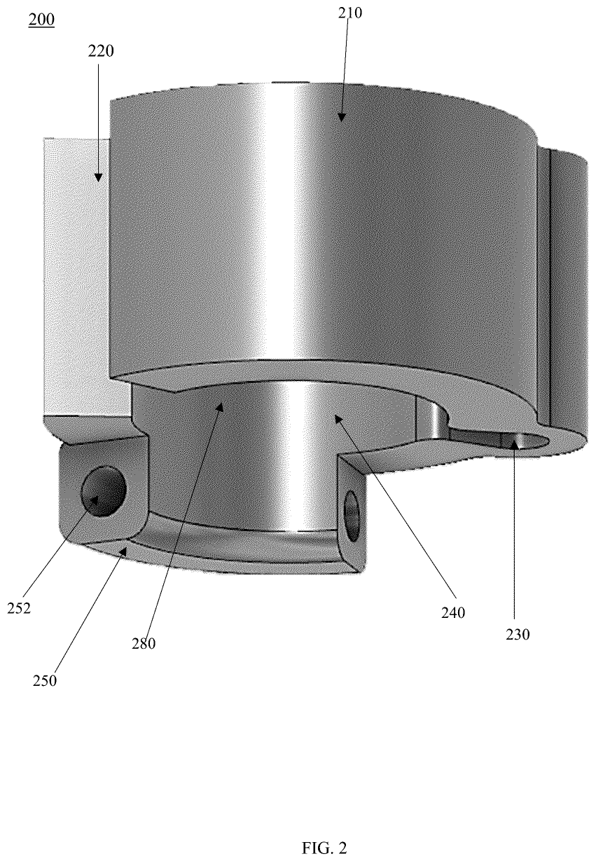

As shown in FIGS. 1 and 2, the retention member 200 may include a body 210. In some examples, the body 210 may have U-shape with an opening 220. In some examples, the center of the body 210 may be aperture 240 in which a medical article (e.g., catheter) may be disposed. In some examples, the body 210 may include a hinge 230 disposed opposite of the opening 220. In some examples, the hinge 230 may be configured to expand the body 210 to increase the diameter of the aperture 240, for example, to enable locking the body 210 around a medical article.

In some examples, the member 200 may include a fastener member 260 disposed to surround an outer surface and the opening 220 of the body 210. In some examples, the member 200 may include a locking member 270 disposed on the body 210 and configured to lock a position of the fastener member 260 with respect to the body 210. In some examples, as shown in FIG. 1, the fastener member 260 may be a gear rack arm and the locking member 270 may be a locking buckle configured to lock a position of the fastener member 260. In other examples, a different locking member 270 and/or a different fastener member 260 may be used. In use, after a medical article is provided within the aperture 240 of the member 200, the clinician may pull the fastener member 260 through the locking member 270 until the body 210 is secured around the article.

In some examples, the inner diameter of the body 210 may be lined with a friction member 280, such as gel adhesive. The friction member 280 may be configured to increase the friction between the member 200 and the medical article, for example, to help prevent the member 200 from slipping.

In some examples, the member 200 may include an attachment member 250 disposed on the body 210, such as the bottom of the body 210, and configured to attach the member 200 to the expandable support frame 300. In some examples, the attachment member 250 may include an extruded hole 252 through which a portion (e.g., a portion of an upper member 340) of the expandable frame 300 may be disposed.

In some examples, the member 200 may be made of one or more polymer materials including but not limited to a thermoplastic monomer, such as polylactic acid (PLA).

In some examples, the expandable support frame 300 may be configured to move from a resting collapsed, flat state (i.e., lays substantially flat in a singular plane) as shown in FIG. 3 to an expandable state as shown in FIG. 1. The expandable support frame 300 may be height-adjustable.

In some examples, the expandable support frame 300 may include a plurality of members. The members may include a base member 320, an upper member 340 and a plurality of middle members 330 disposed between the base member 320 and the upper member 340.

In some examples, the expandable support frame 300 may be configured to linearly extend to an expanded state from a collapsed state with respect to the base member 320 so that the plurality of middle members 330 and the upper member 340 are disposed above the base member 320 when in the expanded state. The height of the support frame 300 may be adjusted to a desired height by adjusting the linear extension of the members. In some examples, the maximum height of the frame 300 may be limited so that it does not exceed the length of the medical article. This may prevent the medical article from accidentally being dislodged, for example, when expanding the frame 300.

In some examples, the expandable support frame 300 may include a first end 302, a second end 304, and a length therebetween. In some examples, the frame 300 may be a wireframe, such as a coil along its length. In some examples, the expandable support frame 300 may include one or more malleable metal materials, such as aluminum.

In some examples, the expandable support frame 300 may have a U-shape with a central aperture 350. The aperture 350 may align with the aperture 240 of the member 200. In some examples, the plurality of members may be semi-concentric rings with an opening 360. The opening 360 may align with the opening 220 of the member 200. In some examples, the plurality of members may wind around the circumference of the member 200 so as to provide the U-shape.

In some examples, each of the plurality of the members may have a different diameter. The diameter of the members may incrementally decrease from the base member 320 to the upper member 340. In some examples, the base member 320 may have the largest diameter so that the diameter of the base member 320 is larger than the diameter of the upper member 340 and a diameter of each middle member 330. The upper member 340 may have the smallest diameter so that the diameter of the upper member 340 is smaller than the diameter of each middle member 330 and the diameter of the base member 320.

In some examples, the base member 320 and/or the upper member 340 may include single wire as shown in the figures. In other examples, the base member 320 and/or the upper member 340 may have a different configuration, such as a double wire.

In some examples, the upper member 340 may be attached to the retention member 200, for example, using the attachment member 250. As shown in FIG. 1, the upper member 340 may be disposed through the extruded hole 252.

In some examples, the anchor member 150 may be attached to the base member 320. In some examples, the base member 320 may be sandwiched between one or more of the layers of the anchor member 150.

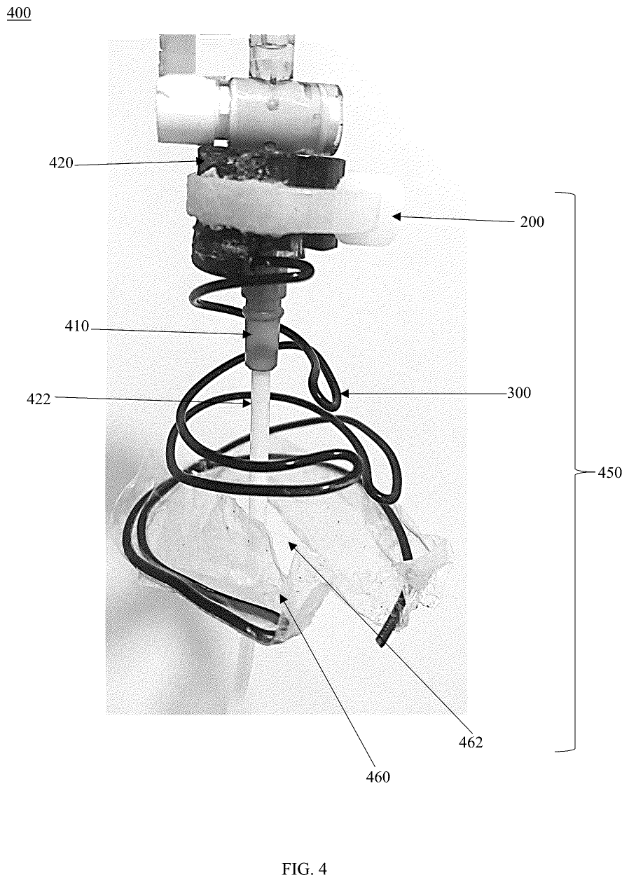

In some examples, the anchor member 150 may have a shape and diameter larger than the diameter of the base member 320. In some examples, the anchor member 150 may have a circular shape as shown in FIG. 1. In some examples the anchor member 150 may have a different shape, for example, as shown in FIG. 4.

FIG. 4 shows an example 400 of a catheter retention device 450 with a different anchor member 460 affixed to a paracentesis catheter 410. As shown in FIG. 4, the retention device 450 may include an anchor member 460 configured to removably adhere to skin of a patient at an area of insertion of a medical article (e.g., abdomen of the patient); a retention member 200 configured to securely hold and surround a medical article (affixed to a region 420 of the catheter 410), such as a catheter (e.g., 410); and an expandable support frame 300 configured to move from a collapsed, resting, state, for example, as shown in FIG. 3, to an expanded state, for example, as shown in FIG. 4. As shown in this example, the retention member 200 can be affixed to the catheter 410 below the stopcock 420. In this example, the anchor member 460 may have a semi-concentric shape that has an opening 462 that corresponds to the openings 360 and 220. In this example, the drainage tubing 422 of the catheter 410 may be disposed within the opening 462.

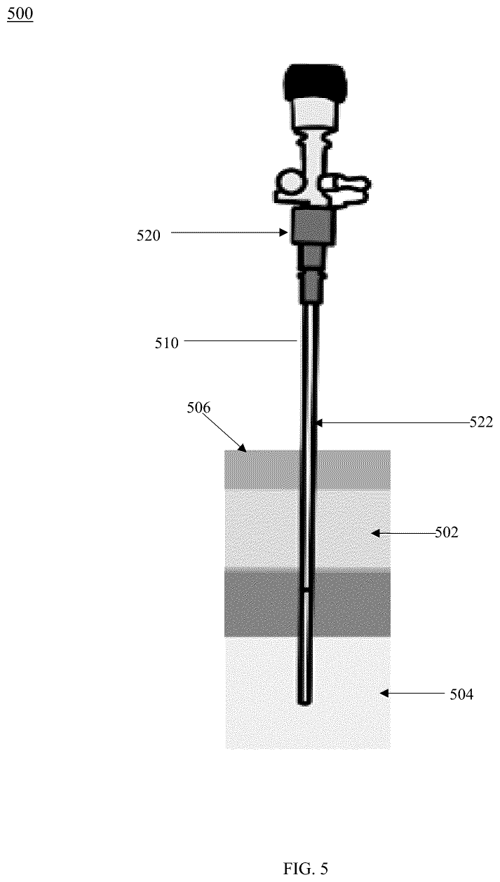

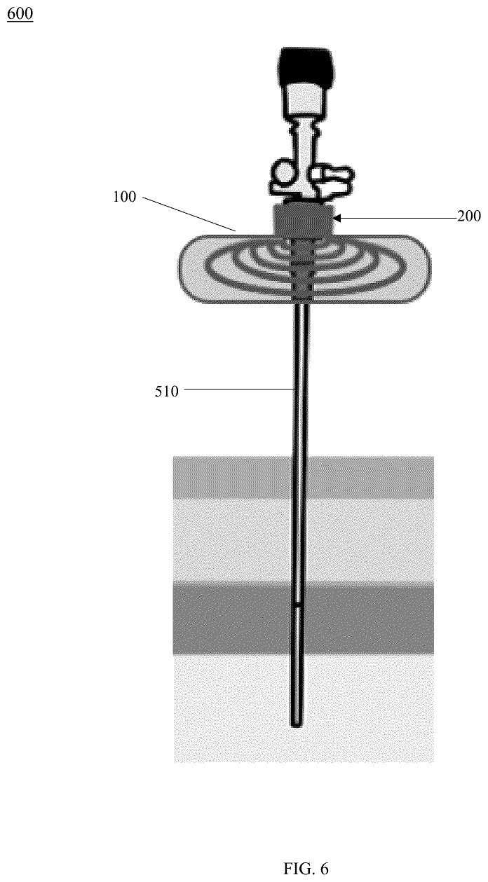

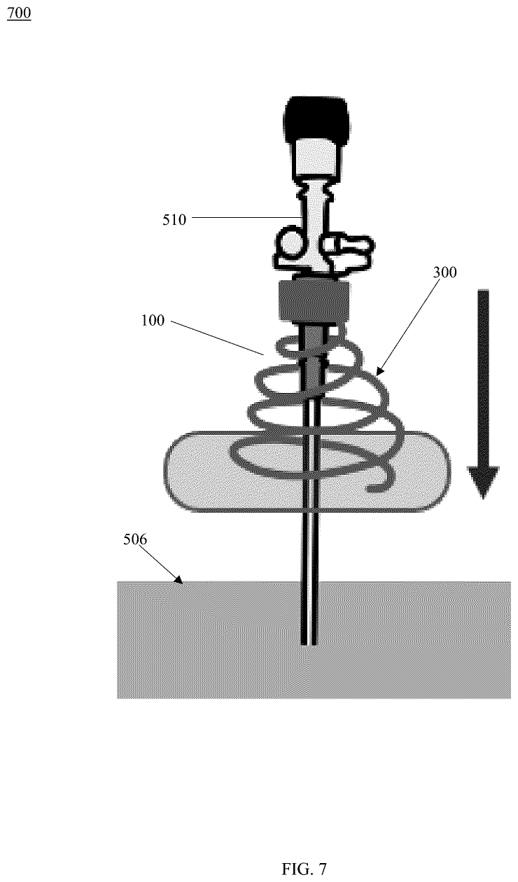

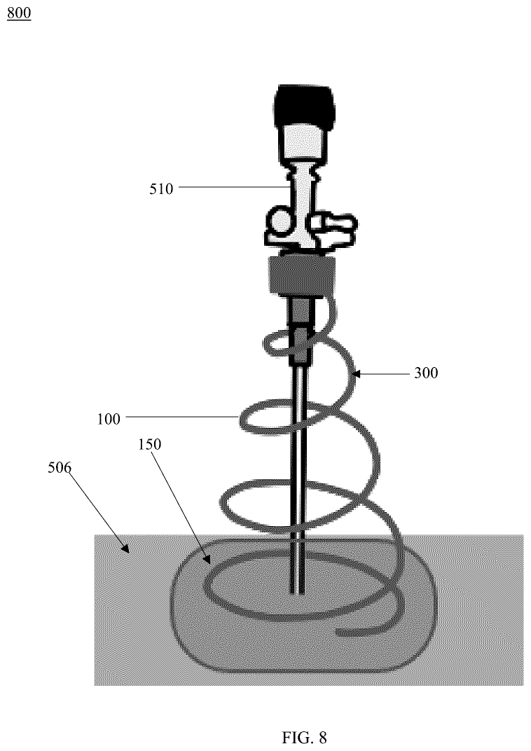

FIGS. 5-8 shows an illustrative example of a paracentesis procedure of using the catheter retention device 100 according to embodiments. A paracentesis catheter 510, like the catheter 410, can be placed at the desired angle and depth with respect to the tissue 502 to access the fluid 504 as shown in example 500 in FIG. 5. After which, a clinician can position the retention device 100 under the stopcock 520 of the catheter 510, like the example shown in FIG. 4, as shown in FIG. 6. The member 200 may be placed around the area below the stopcock 520 (e.g., the plastic) of the catheter 510. The clinician may feed the fastener 260 through the locking arm 270 to a desired position to tighten the member 200 around the catheter 510, like the example shown in FIG. 4, as shown in FIG. 6. The position of the member 200 does not interfere with the three-way stopcock 520 that connects the catheter 510 to drainage tubing 522; therefore addressing the user need for compatibility with existing drainage mechanisms. After the member 200 is secured around the catheter 510 and the tubing 222 is placed through the anchor member 150, the clinician can expand the frame 300, as shown in FIG. 7, by either moving the base member 320 away from the upper member 340 and/or moving the upper member 340 away from base member 320. Before and/or after the expansion of the frame 300, the backing material may be removed from the bottom face of the anchor member 150. After the frame 300 is expanded to the desired height, the anchor member 150 may be interfaced with/adhered to the patient's skin 506 via its bottom face, as shown in FIG. 8. After the device 100 and the retained catheter 510 are secured to the patient's abdomen, the procedure (e.g., fluid removal) can begin. When the paracentesis procedure has been completed, the anchor member 150 can be detached from the patient's skin 506 to remove the device 100 and the retained catheter 510.

The features, such as the combination of the retention member 200 and the support frame 300, of the disclosed devices can allow the disclosed devices to be placed onto a medical article, such as a paracentesis catheter, after insertion, preventing the catheter retention device from hindering the insertion procedure, and therefore addressing the user need of mitigation of failure and correction. In addition, the adjustable nature of the support frame 300 can allow the catheter retention device to accommodate a large range of patients, as each patient has a different abdomen morphology and may require more or less depth of insertion. Thus, the disclosed devices can allow a clinician to fixate the catheter at constant depth and insertion angle without the need to hold the catheter in place, ensuring hands-free retention to fulfill the user need for stability and retention.

It will be understood that the device may be used with other medical articles and procedures and is not limited to the paracentesis catheter and paracentesis procedure as disclosed and shown. For example, it may be used with different catheters, different instruments, among others for different procedures.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed:

1. A catheter retention device comprising:
an expandable support frame having including a plurality of members, the plurality of members including a base member, an upper member and a plurality of middle members disposed between the base member and the upper member, the expandable support frame configured to linearly extend to an expanded state from a collapsed state with respect to the base member so that the plurality of middle members and the upper member are disposed above the base member when in the expanded state;
a retention member disposed on the upper member, the retention member configured to securely hold and surround a medical article; and
an anchor member disposed on the base member, the anchor member including an adhesive layer disposed on a bottom face of the anchor member.

2. The device according to claim 1, wherein the expandable support frame is a wireframe.

3. The device according to claim 1, wherein the expandable support frame has a U-shape.

4. The device according to claim 3, wherein each member is a semi-concentric ring and has a different diameter.

5. The device according to claim 4, wherein a diameter of the base member is larger than a diameter of the upper member and a diameter of each middle member.

6. The device according to claim 5, wherein a diameter of the upper member is smaller than the diameter of each middle member.

7. The device according to claim 3, wherein the expandable support frame has an opening that corresponds to an opening of the retention member.

8. The device opening according to claim 7, wherein the expandable support frame has an aperture that corresponds to an aperture of the retention member, the medical article being configured to be disposed within the aperture.

9. The device according to claim 1, wherein in the collapsed state, the expandable support frame is substantially flat.

10. The device according to claim 1, wherein the anchor member has a U-shape.

11. The device according to claim 1, wherein the retention member includes:
a body having an aperture configured to receive the medical article, the body having a U-shape with an opening;
wherein the body includes a hinge disposed opposite of the opening; and
a fastener member disposed to surround an outer surface and the opening of the body.

12. The device according to claim 11, wherein the retention member includes a locking member configured to lock a position of the fastener member.

13. The device according to claim 12, wherein the locking member is configured to secure the medical article within the retention member.

14. The device according to claim 12, wherein the retention member includes a friction member disposed along one or more portions of an inner surface of the body.

15. The device according to claim 1, wherein the medical article is a paracentesis catheter.

16. The device according to claim 1, wherein the adhesive is configured to removably adhere to skin of a patient at the treatment site.
